(19) United States
(12) Patent Application Publication
(10) Pub. No.: US 2011/0171147 A1
(43) Pub. Date: Jul. 14, 2011

(54) Method for the Production of Unsaturated Alpha, Omega Dicarboxylic Acids and/or Unsaturated Alpha, Omega Dicarboxylic Diesters

(75) Inventors: Markus Samorski, Duesseldorf (DE); Markus Dierker, Duesseldorf (DE)

(73) Assignee: COGNIS IP MANAGEMENT GMBH, Duesseldorf (DE)

(21) Appl. No.: 13/060,011

(22) PCT Filed: Aug. 12, 2009
(86) PCT No.: PCT/EP2009/005841

§ 371 Date: Apr. 5, 2011

(30) Foreign Application Priority Data
Aug. 21, 2008 (EP) 08014838

(51) Int. Cl. A61K 8/18; C07C 69/52; A61Q 17/04

(52) U.S. Cl. 424/59; 560/205; 560/225

(57) ABSTRACT

The invention relates to a method for the production of unsaturated alpha,omega-dicarboxylic acids and/or their corresponding diesters, by reacting unsaturated carboxylic acids and/or their corresponding esters under the influence of specific ruthenium complex catalysts. The unsaturated alpha,omega-dicarboxylic acids and/or alpha,omega-dicarboxylic acid diesters obtained by the method are useful as UV filters in cosmetic sun screen preparations.

The present invention relates to a method for the production of unsaturated alpha,omega dicarboxylic acids, and alpha,omega dicarboxylic diesters which have improved absorption in the UV region, in particular in the UV-B region.

For sunscreen preparations, there is a constant need to improve the UV absorption and thus the UV protection of skin and hair. Here, in particular, the absorption of short-wave, high-energy UV-B radiation (280-320 nm) is of importance since this can lead not only to photoerythemas, sunburn and skin necroses, but also to skin cancer (melanomas). However, classic UV photoprotective filter substances present the person skilled in the art with challenges as regards formulation, and intolerances of the skin to UV photoprotective filter substances are also known.

The object of the present invention was to provide substances which have improved absorption in the UV region, in particular in the UV-B region, and can be used instead of or in addition to known UV photoprotective filter substances. These substances should be able to be formulated with the customary cosmetic ingredients.

Furthermore, it was of interest to obtain substances that are as little colored as possible and which are suitable for incorporation in colorless or transparent cosmetic preparations.

Surprisingly, it has been found that, using the method according to the invention, unsaturated alpha,omega dicarboxylic acids and alpha,omega dicarboxylic diesters are obtained which have an increased absorption in the UV region, in particular in the UV-B region. Unsaturated alpha,omega dicarboxylic acids and/or alpha,omega dicarboxylic diesters obtained by the process according to the invention are therefore suitable in particular in cosmetic preparations for sun protection.

Methods for the production of unsaturated alpha,omega dicarboxylic diesters starting from unsaturated dicarboxylic acids are described in the prior art: Ngo et al. (JAOCS, Vol 83, No. 7, pp. 629-634, 2006) describes the metathesis of unsaturated carboxylic acids with the help of the so-called Grubbs catalysts of the first and second generation. The method with Grubbs catalysts of the first generation is described in U.S. Pat. No. 5,750,815. J. Mol. (Topics in Catalysis, Vol. 27, Nos. 1-4, pp. 97-104, February 2004) and A. Rybak et al. (Green Chem. 2007, 9, 1356-1361) also describe such methods.

The invention provides a method for the production of unsaturated alpha,omega dicarboxylic acids and alpha,omega dicarboxylic diesters in which unsaturated carboxylic acids and/or esters of unsaturated carboxylic acids are reacted in the presence of catalyst (1) and/or (2)

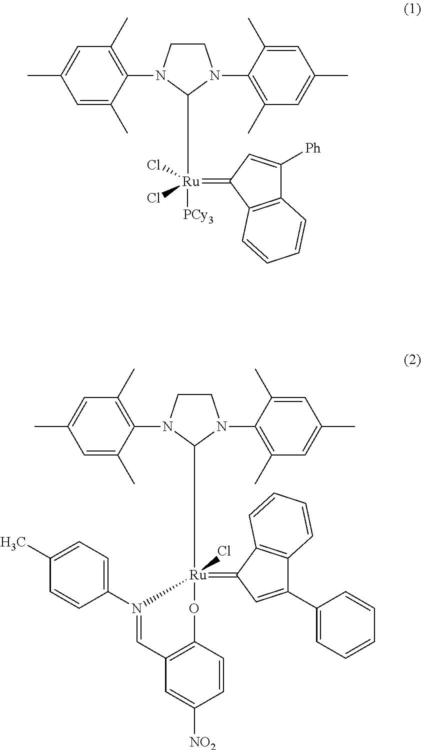

### Starting Materials

Suitable starting materials for the method according to the invention are mono- or polyunsaturated carboxylic acids having 14 to 24 carbon atoms, which may be optionally branched. The double bonds of the carboxylic acids here may be present either in the cis or in the trans configuration.

Suitable carboxylic acids are the compounds of the following formula R₁—COOH, where R₁ is a mono- or polyunsaturated alkyl radical having 13 to 23 carbon atoms. Polyunsaturated alkyl radicals which may be mentioned are, for example, di-, tri-, tetra-, penta- or hexaunsaturated alkyl radicals.

The R₁ radical may be cyclic or acyclic, the radical R₁ preferably being acyclic. The R₁ radical may be branched or unbranched, preference being given to carboxylic acids with an unbranched radical R₁. Particular preference is given to carboxylic acids with a noncyclic radical R₁.

The following shorthand is used for describing the unsaturated carboxylic acids: the first number describes the total number of carbon atoms in the carboxylic acids, the second number describes the number of double bonds, and the number in brackets describes the position of the double bond relative to the carboxylic acid group. Consequently, the shorthand for oleic acid is 18:1 (9). If the double bond is in the trans configuration, this is denoted by the abbreviation “tr”. Consequently, the shorthand for elaidic acid is 18:1 (tr9).

Suitable monounsaturated carboxylic acids are, for example, myristoleic acids [14:1 (9), (9Z)-tetradeca-9-enoic acid], palmitoleic acid [16:1 (9); (9Z)-hexadeca-9-enoic acid], petroselic acid [(6Z)-octadeca-6-enoic acid], oleic acid [18:1 (9); (9Z)-octadeca-9-enoic acid], elaidic acid [18:1 (tr9); (9E)-octadeca-9-enoic acid)], vaccenic acid [18:1 (tr11); (11E)-octadeca-11-enoic acid], gadoleic acid [20:1 (9); (9Z)-eicosa-9-enoic acid], eicosenoic acid (=gondoic acid) [20:1 (11); (11Z)-eicosa-11-enoic acid], cetoleic acids [22:1 (11); (11Z)-docosa-11-enoic acid], erucic acid [22:1 (13); (13Z)-docosa-13-enoic acid], nervonic acid [24:1 (15); (15Z)-tetracosa-15-enoic acid].

Suitable polyunsaturated carboxylic acids are, for example, linoleic acid [18:2 (9,12); (9Z-12Z)-octadeca-9,12-dienoic acid], alpha-linolenic acid [18:3 (9,12,15); (9Z,12Z,15Z)-octadeca-9,12,15-trienoic acid], gamma-linolenic acid [18:3 (6,9,12); (6Z,9Z,12Z)-octadeca-6,9,12-trienoic acid], calendic acid [18:3 (8,10,12); (8E,10E,12Z)-octadeca-8,10,12-trienoic acid], punicic acid [18:3 (9,11,13); (9Z,11E,13Z)-octadeca-9,11,13-trienoic acid], alpha-eleostearic acid [18:3 (9,11,13); (9Z,11E,13E)-octadeca-9,11,13-trienoic acid], arachidonic acid [20:4 (5,8,11,14), (5Z,8Z,11Z,14Z)-eicosa-5,8,11,14-tetraenoic acid], timnodonic acid [20:5 (5,8,11,14,17), (5Z,8Z,11Z,14Z,17Z)-eicosa-5,8,11,14,17-pentaenoic acid], clupandodonic acid [22:5 (7,10,13,16,19), (7Z,10Z,13Z,16Z,19Z)-docosa-7,10,13,16,19-pentaenoic acid], cervonic acid [22:6 (4,7,10,13,16,19), (4Z,7Z,10Z,13Z,16Z,19Z)-docosa-4,7,10,13,16,19-hexaenoic acid].

Suitable starting materials are also esters of the specified mono- or polyunsaturated carboxylic acids. Suitable esters are in particular esters of these carboxylic acids with alcohols R₂—OH, where R₂ is an alkyl radical having 1 to 8 carbon atoms. Suitable radicals R₂ which may be mentioned are, for example: methyl, ethyl, propyl, isopropyl, butyl, 2-methylpropyl, pentyl, 2,2-dimethylpropyl, 2-methylbutyl, 3-methylbutyl, hexyl, 1-methylpentyl, 2-methylpentyl, 3-methylpenyl, 4-methylpentyl, 1-ethylbutyl, 2-ethylbutyl, heptyl and octyl radicals.

Suitable starting materials are also esters of the specified mono- or polyunsaturated carboxylic acids with glycerol (=glycerol esters). Of suitability here are both glycerol monoesters (=monoglycerides, monoacylglycerol), glycerol diesters (=diglycerides, diacyl glycerol) and also glycerol triesters (=triglycerides, triacylglyceryl) and also mixtures of these various glycerol esters.

The unsaturated carboxylic acids or the esters of the unsaturated carboxylic acids may be present either individually or else in mixtures with one another. If exclusively one unsaturated carboxylic acids or the ester of only one unsaturated carboxylic acids is used, then a reaction referred to as self-metathesis takes place. If different unsaturated carboxylic acids or esters of different unsaturated carboxylic acids are used, a reaction referred to as cross-metathesis takes place.

In one preferred embodiment of the invention, monounsaturated carboxylic acids and/or esters of monounsaturated carboxylic acids and/or mixtures of the monounsaturated carboxylic acids or mixtures of the esters of monounsaturated carboxylic acids are used.

### Catalyst

The method according to the invention is carried out in the presence of catalysts (1) and/or (2).

Catalyst (1) is the compound of the following structural formula:

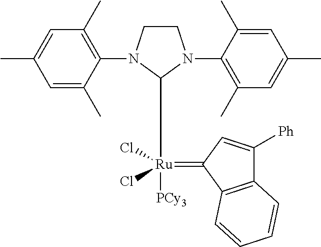

The chemical name for this catalyst is dichloro[1,3-bis(mesityl)-2-imidazolidinylidene]-(3-phenyl-1H-inden-1-ylidene)(tricyclohexylphosphine)ruthenium(II), CAS No. 536724-67-1. It is commercially available, for example, under the name Neolyst™M2 from Umicore.

Catalyst (2) is the compound of the following structural formula:

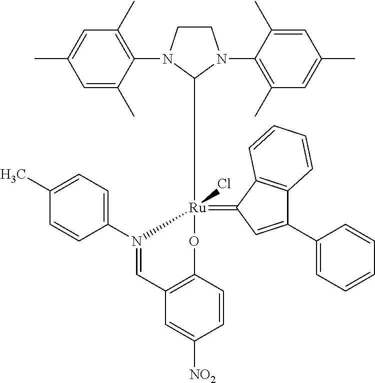

The chemical name for this catalyst is [1,3-bis(2,4,6-trimethylphenyl)-2-imidazolidinylidene]-[2-[[(4-methylphenyl)imino]methyl]-4-nitrophenolyl]-[3-phenyl-1H-inden-1-ylidene]ruthenium(II) chloride, CAS No. 934538-04-2. It is commercially available, for example, under the name Neolyst™M41 from Umicore.

The catalyst according to the invention is preferably used in concentrations of less than or equal to 1000 ppm, in particular in the range from 10 to 1000 ppm, preferably 20 to 200 ppm.

### Reaction Conditions

The method according to the invention can be carried out at temperatures of from 0 to 100° C., preferably at 20 to 90° C., in particular at 40 to 80° C.

The method can in customary solvents in which the starting materials and the catalyst dissolve; for example, mention may be made of: hydrocarbons or alcohols. In one preferred embodiment of the invention, the method can be carried out without a solvent.

### Purification

The method according to the invention produces unsaturated alpha,omega dicarboxylic acids and unsaturated alpha,omega dicarboxylic diesters, and also the corresponding unsaturated hydrocarbons. The mixture can be separated e.g. by distillation, by fractional crystallization or by extraction.

If desired, the products obtained in this way can be subjected to a hydrogenation.

### Use

Surprisingly, it has been found that the unsaturated alpha,omega dicarboxylic acids and also unsaturated alpha,omega dicarboxylic diesters produced by the method according to the invention are suitable for use in cosmetic preparations. On account of their UV-B absorption, they are suitable in particular in cosmetic preparations for sun protection.

### EXAMPLES

### Example 1a

50 ml of a methyl ester mixture (consisting of 92.0% methyl oleate and 2.9% methyl linoleate, percentages refer to a non-calibrated area percent GC method) were reacted for 1.4 h at 50° C. in the presence of 200 ppm of catalyst (1) [CAS No. 536724-67-1]. After the reaction had ended, 23.1% of dimethyl diester and 20.2% of 9-octadecene were obtained.

### Example 1b

Example 1a) was repeated, except that the reaction was carried out at 40° C. for 4 h. The yield was identical to example 1a).

### Example 1c

Example 1a) was repeated, except that the reaction was carried out at 30° C. for 7 h. The yield was identical to example 1a).

### Example 2

50 ml of a methyl ester mixture (consisting of 92.0% methyl oleate and 2.9% methyl linoleate) were reacted for 8 h at 80° C. in the presence of 200 ppm of catalyst (2) [CAS No. 934538-04-2]. After the reaction had ended, 23.3% of dimethyl diester and 20.6% of 9-octadecene were obtained.

### Example 3

10 g of high oleic sunflower methyl ester (with a content of 75 to 93% methyl oleate) were weighed into a 50 ml 2-neck round-bottom flask and admixed with 0.2 g of the ruthenium catalyst Neolyt™ M2 from Umicore [CAS No.: 536724-67-1] (=catalyst (1)). The flask was then placed under nitrogen blanketing into a Petri dish filled with water and standing on a magnetic stirrer, and stirring was carried out for 60 min at 35-40° C. After cooling to room temperature, the catalyst solid was filtered through a column filled with silica gel 60. This gave a red-brown liquid.

### Example 4

### Comparative Example

10 g of high oleic sunflower methyl ester (with a content of 75 to 93% methyl oleate) were weighed into a 50 ml 2-neck round-bottom flask and admixed with 0.2 g of the ruthenium catalyst dichloro(3-methyl-2-butenylidene)bis(tricyclohexylphosphine)ruthenium(II) [commercially available from Aldrich, catalog number 576881, CAS No. 194659-03-5]. The flask was then placed under nitrogen blanketing into a Petri dish filled with water and standing on a magnetic stirrer, and stirring was carried out for 60 min at 35-40° C. After cooling to room temperature, the catalyst solid was filtered through a column filled with silica gel 60. This gave a red-brown liquid.

### UV Absorption:

The UV-B absorption of the product obtained according to example 3 and also of the product obtained according to example 4 was measured. Surprisingly, the product obtained according to example 3 exhibited a higher absorption in the range from 240 to 280 nm compared to the product produced in accordance with the prior art (example 4).

1. A method for the production of unsaturated alpha,omega dicarboxylic acids and/or alpha,omega dicarboxylic diesters, in which unsaturated carboxylic acids and/or esters of unsaturated carboxylic acids are reacted in the presence of the catalyst (1) and/or (2)

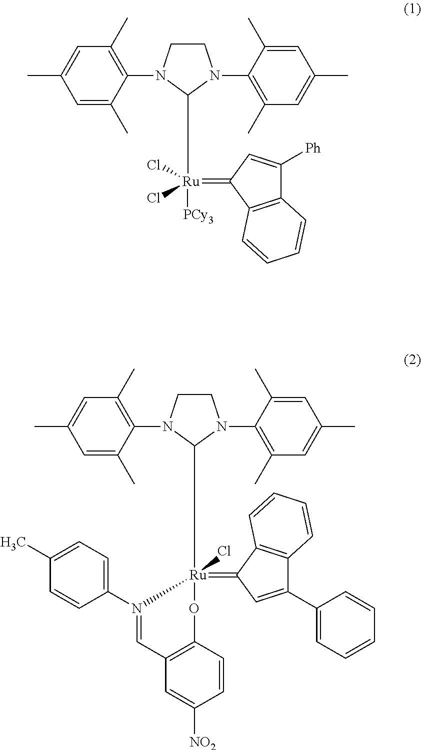

2. The method as claimed in claim 1, characterized in that unsaturated C14 to C24 carboxylic acids are used.

3. The method as claimed in claim 1, characterized in that esters of unsaturated carboxylic acids are used, where the unsaturated carboxylic acid is a C14 to C24 carboxylic acid.

4. The method as claimed in claim 3, characterized in that an ester of C1 to C8 alcohols with unsaturated carboxylic acids and/or an ester of glycerol with unsaturated carboxylic acids is used as ester.

5. The method as claimed in claim 4, characterized in that the glycerol ester is selected from the group consisting of glycerol monoester, glycerol diester and glycerol triester.

6. The method as claimed in one of the preceding claims, characterized in that monounsaturated carboxylic acids are used.

7. The method as claimed in one of the preceding claims, characterized in that the catalyst is used in an amount of from 10 to 1000 ppm, preferably from 20 to 200 ppm.

8. The method as claimed in one of the preceding claims, characterized in that the reaction is carried out at 0 to 100° C., preferably at 20 to 90° C., in particular at 40 to 80° C.

9. The method as claimed in one of the preceding claims, characterized in that the procedure is solvent-free.

10. The method as claimed in one of the preceding claims, characterized in that the unsaturated alpha,omega dicarboxylic acids and/or alpha,omega dicarboxylic diesters and unsaturated hydrocarbons are separated by means of distillation.

11. The use of the unsaturated alpha,omega dicarboxylic acids and/or alpha,omega dicarboxylic diesters obtainable by the method of claims 1 to 10 in cosmetic preparations, in particular in cosmetic preparations for sun protection.
